(19) United States
(12) Patent Application Publication
(10) Pub. No.: US 2025/0186483 A1
(43) Pub. Date: Jun. 12, 2025

(54) METHODS AND COMPOSITIONS FOR PROMOTING COGNITIVE FUNCTIONING

(71) Applicant: TAIYO KAGAKU Co., Ltd., Yokkaichi-shi (JP)

(72) Inventors: Aya ABE, Yokkaichi-shi (JP); Makoto OZEKI, Yokkaichi-shi (JP)

(21) Appl. No.: 18/976,565

(22) Filed: Dec. 11, 2024

(30) Foreign Application Priority Data
Dec. 12, 2023 (JP) 2023-209493

(51) Int. Cl. A61K 31/736; A61P 25/28

(52) U.S. Cl. CPC A61K 31/736; A61P 25/28

(57) ABSTRACT

A partially hydrolyzed guar gum (PHGG) for promoting cognitive functioning has an average molecular weight of 1.0×10³ to 2.0×10⁵, with 70% or more by mass of the components thereof falling within this range, and a 1% by mass aqueous solution of the PHGG has a viscosity, measured at 25° C. and 60 rpm using a B-type viscometer, of 50 mPa·s or less. The PHGG may be produced by hydrolyzing galactomannan polysaccharide obtained from guar endosperm and having a galactose to mannose content ratio (galactose:mannose) in the range of 1:1.3 to 1:2.1, using β-mannanase to reduce the molecular weight. The PHGG contains at least 70% by mass of dietary fiber as determined by enzymatic HPLC.

### CROSS-REFERENCE

The present application claims priority to Japanese patent application no. 2023-209493 filed on Dec. 12, 2023, the contents of which are incorporated herein by reference.

### TECHNICAL FIELD

The invention generally relates to compositions of matter and to methods for promoting cognitive functioning, which are intended, e.g., for healthy elderly people (particularly those age 60 or older).

### BACKGROUND ART

Cognitive functioning is a general term for the intellectual processes and functions required for “cognition,” which encompasses all aspects of intellectual processes and functions, such as perception, judgment, imagination, inference, decision-making, memory, and language comprehension. It refers to the ability to recognize the current situation, use language, and remember, learn, calculate, and predict, based on information obtained from the outside world through one or more of the five senses (sight, hearing, smell, taste, and touch).

Dementia is known as a decline in cognitive functioning. Dementia occurs due to the death and decline of function of brain cells caused by aging, and symptoms gradually become more severe in proportion to the degree of decline in cognitive functioning. Even if it does not lead to dementia, it is inevitable that cognitive functioning will decline to some extent due to aging.

Research on improving cognitive functioning or suppressing the decline of cognitive functioning has been performed; for example, JP 2023-111729 discloses the effects of aged garlic extract.

Furthermore, although a method for reducing stress disorders using fermentable fibers, such as, e.g., partially hydrolyzed guar gum, was suggested in WO 2021/105338, no suggestion or evidence of a cognitive functioning effect by partially hydrolyzed guar gum was provided therein.

### SUMMARY OF THE INVENTION

It is therefore one non-limiting object is to disclose techniques for promoting cognitive functioning.

The inventors have found that a composition containing a specific partially hydrolyzed guar gum can promote cognitive functioning, in particular in elderly subjects (particularly those age 60 or older).

Thus, in one aspect of the present teachings, a method for promoting cognitive functioning, e.g., of a healthy subject, may comprise administering, preferably enterally, more preferably orally, to a human in need thereof a partially hydrolyzed guar gum (PHGG) having an average molecular weight of 1.0×10³ to 2.0×10⁵, wherein 70% or more by mass of the components of the PHGG fall within that range, and wherein a 1% by mass aqueous solution of the partially hydrolyzed guar gum has a viscosity, measured at 25° C. and 60 rpm using a B-type viscometer, of 50 mPa's or less. The partially hydrolyzed guar gum is preferably produced by hydrolyzing galactomannan (polysaccharide), which is derived from guar endosperm and has a galactose to mannose content ratio (galactose:mannose) in the range of 1:1.3 to 1:2.1, using a β-mannanase, e.g., a microbial β-mannanase, to reduce the molecular weight (partially hydrolyze), such that the PHGG preferably contains at least 70% by mass of dietary fiber as determined by enzymatic HPLC. Herein, “HPLC” means high-performance liquid chromatography.

In the present aspect, cognitive functioning is preferably one or more processes or functions selected from visual memory, composite memory, verbal memory, psychomotor speed, reaction time, cognitive flexibility, and executive function. Particularly preferably, the present PHGG compositions promote visual memory. As described above, cognitive functioning includes intellectual processes and functions such as perception, judgment, imagination, inference, decision-making, memory, and language comprehension. It has been found that compositions of matter of the present disclosure are particularly effective at improving visual memory, composite memory, verbal memory, psychomotor speed, reaction time, cognitive flexibility, and executive function.

In another aspect of the present teachings, a food, drink or pharmaceutical product containing a cognitive function promotor can be provided. Food and drink products according to the present teachings include, but are not limited, various foods and beverages, including, e.g., nutritional supplements, health foods, foods for specified health uses, functional foods, dietary foods, general health foods, supplements, tea, coffee, juices, soft drinks, energy drinks, cooked rice, bread, noodles, dairy products, processed egg products, processed seafoods, livestock processed foods, confectionery, oils and fats, oils and fats processed foods, seasonings, and side dishes. Pharmaceutical products according to the present teachings include, but are not limited to, medicines or quasi-drug products, such as oral preparations and enteral preparations, and can be in the form of liquids, tablets, granules, pills, syrups, etc.

Thus, according to the present teachings, techniques for promoting cognitive functioning are provided.

### BRIEF DESCRIPTION OF THE DRAWINGS

FIG. 1 is a bar graph comparing standardized score data for 16 cognitive domains (neurocognition index (NCI), composite memory, verbal memory, visual memory, psychomotor speed, reaction time, complex attention, cognitive flexibility, processing speed, executive function, social acuity, reasoning, working memory, sustained attention, simple attention, motor speed) before the start of the study (0w) and after the end of the study (12w) within the test food group (PHGG group), for 8 data points where significant differences were observed before and after the study. In FIG. 1, “*” indicates that a significant difference was observed at p<0.05 and “**” indicates that a significant difference was observed at p<0.01.

FIG. 2 is a bar graph showing the results of a visual memory evaluation performed in the manner described with regard to FIG. 1 above. In the graph, the value of p indicates the significance level, meaning that p=0.023 was observed between the placebo group (Placebo) and the subject group (PHGG) after 12 weeks.

### DETAILED DESCRIPTION OF PREFERRED EMBODIMENTS

Next, embodiments of the present teachings will be described with reference to the drawings. However, the technical scope of the invention is not limited to these embodiments, and the invention can be embodied in various forms without changing the gist of the invention.

“Guar gum” refers to a water-soluble, naturally occurring polysaccharide obtained from the endosperm of guar beans (specifically, cotyledons), which is a polysaccharide having a side chain of one galactose molecule attached to two linearly bonded mannose molecules and which has an average molecular weight of about 2.0×10⁵ to 3.0×10⁵. Guar gum is known to have physiological effects such as suppressing an increase in blood sugar level, lowering cholesterol, and improving bowel movements. Herein, the term “partially hydrolyzed guar gum” refers to a water-soluble dietary fiber obtained by partially hydrolyzing and thereby decreasing the average molecular weight of the galactomannan polysaccharide contained in the endosperm of guar (scientific name: Cyanopsis tetragonoloba), an annual legume plant native to India, Pakistan, etc., which is used as a raw material. Methods for hydrolyzing guar gum are not particularly limited, and may include enzymatic decomposition, acidic decomposition, or the like. Enzymatic decomposition is preferable because it is easy to adjust the molecular weight of the decomposition (hydrolyzed) products.

The enzyme(s) used in enzymatic hydrolysis is (are) not particularly limited as long as the enzyme(s) hydrolyze(s) a linear mannose chain. However, it is preferable to use β-mannanase derived from Aspergillus or Rhizopus. The upper limit of the average molecular weight distribution of the partially hydrolyzed guar gum according to the present teachings is preferably 2.0×10⁵ or less, e.g., 1.0×10⁵ or less, e.g., 2.5×10⁴ or less. The lower limit of the average molecular weight distribution of the partially hydrolyzed guar gum is preferably 1.0×10² or more, e.g., 2.0×10³ or more. If the average molecular weight exceeds 2.0×10⁵, the viscosity becomes too high and it becomes difficult to integrate the partially hydrolyzed guar gum into foods and drinks. Methods for measuring the molecular weight distribution are not particularly limited, but suitable methods include, for example, gel filtration chromatography that uses polyethylene glycols (average molecular weights: 2×10², 2×10³, 2×10⁴ and 1×10⁵) as molecular weight markers.

Partially hydrolyzed guar gum (PHGG) according to the present teachings preferably contains at least 70% by mass, preferably at least 80% by mass, of components (partially hydrolyzed polysaccharides) falling within the above-mentioned average molecular weight range.

Galactose is a type of monosaccharide classified as an aldohexose, and has the molecular formula C₆H₁₂O₆ and a molecular weight of 180 g/mol (both are the same as glucose). In the chemical structure of galactose, the hydroxyl groups (—OH) at the 2nd position (second from the top in the Fischer projection) and 5th position point (extend) in the same direction, while those at the 3rd and 4th positions are in the opposite direction, and the structure at the 5th position of D-galactose is the same as that of D-glyceraldehyde.

Mannose is a type of monosaccharide that is also classified as an aldohexose, and also has the molecular formula C₆H₁₂O₆ and a molecular weight of 180 (both are the same as glucose). In the chemical structure of mannose, the hydroxyl groups (—OH) at the 2nd and 3rd positions point (extend) in the same direction, while those at the 4th and 5th positions are in the opposite direction, and the structure at the 5th position of D-mannose is the same as that of D-glyceraldehyde. Mannose is not metabolized by humans, and when ingested orally, very little of the mannose enters the glycolytic pathway.

“Cognition” refers to processes in which a human being perceives an object in the outside world and then judges or interprets what it is. “Cognitive functioning” refers to the higher processes and functions of the brain, including cognitive functions such as perception, judgment, imagination, inference, decision-making, memory, calculation, understanding, learning, thinking, and language. Elderly people inevitably experience a decline in cognitive functioning compared to younger people. Therefore, healthy elderly people age 60 or older are preferably subjects according to the present teachings, excluding those diagnosed with dementia.

Cognitive function promotors of the present teaching can be taken orally either directly or mixed with food, beverages, etc. The dosage of the cognitive function promotor when orally administered is not particularly limited, but is preferably 0.5 g to 70 g per day per adult (more preferably 3 g to 30 g, and even more preferably 6 g to 18 g).

As mentioned above, cognitive functioning includes many processes and functions, but the present teachings primarily target improving one or more processes or functions selected from visual memory, composite memory, verbal memory, psychomotor speed, reaction time, cognitive flexibility, and executive function.

### Preparation of Partially Hydrolyzed Guar Gum (PHGG)

### Example 1

After adjusting the pH to 4.5 by adding 0.1N hydrochloric acid to 900 g of water, 0.2 g of commercially available β-mannanase derived from Aspergillus bacteria and 100 g of guar gum powder were added and mixed. This mixture was allowed to react at 40° C. to 45° C. for 24 hours. Thereafter, the enzyme was inactivated by heating at 90° C. for 15 minutes. The reaction solution was filtered and separated by suction filtration, and the transparent solution obtained by removing insoluble matter was concentrated under reduced pressure (Yamato evaporator). A solid content of 20% by mass was obtained. This transparent solution was dried using a spray dryer (Okawahara Kakoki Co., Ltd.), yielding 65 g of partially hydrolyzed guar gum (PHGG) as a powder.

The partially hydrolyzed guar gum was dissolved in water to prepare an aqueous solution having a concentration of 0.5% (w/v). The average molecular weight was determined by gel filtration chromatography (column: YMC-Pack Diol-120, detector: differential refractometer) using a set of polyethylene glycols (average molecular weights: 2×10², 2×10³, 2×10⁴ and 1×10⁵) as molecular weight markers, and was found to be approximately 20,000, with more than 80% by mass of the components of the PHGG having molecular weights in the range of 1.0×10³ to 2.0×10⁵.

The viscosity of a 1% by mass aqueous solution of the PHGG was measured using a B-type viscometer at 25° C. and 60 rpm, and was found to be 8 mPa·s.

The galactose to mannose content ratio (galactose:mannose) was measured to be 1:1.7.

The dietary fiber content was measured by enzymatic HPLC and was found to be 90% by mass.

### Example 2

After adjusting the pH to 3 by adding 0.1N hydrochloric acid to 900 g of water, 0.15 g of commercially available β-mannanase derived from Aspergillus bacteria and 100 g of guar gum powder were added and mixed. This mixture was allowed to react at 40° C. to 45° C. for 24 hours. Thereafter, the enzyme was inactivated by heating at 90° C. for 15 minutes. The reaction solution was filtered and separated by suction filtration, and the transparent solution obtained by removing insoluble matter was concentrated under reduced pressure (Yamato evaporator). A solid content of 20% by mass was obtained. This transparent solution was dried using a spray dryer (Okawahara Kakoki Co., Ltd.), yielding 68 g of partially hydrolyzed guar gum as a powder.

The average molecular weight of the guar gum decomposition product was determined in the same manner as in Example 1 and was approximately 2.5×10⁴. The HPLC chart showed that the molecular weight was 1.0×10³ to 2.0×10⁴ for 80% by mass or more of the components of the PHGG.

Furthermore, the viscosity of a 1% by mass aqueous solution was measured using a B-type viscometer at 25° C. and 60 rpm, and was found to be 10 mPa·s.

The galactose to mannose content ratio (galactose:mannose) was measured to be 1:1.8.

The dietary fiber content was measured using enzymatic HPLC, and was found to be 89% by mass.

### Example 3

After adjusting the pH to 4 by adding 0.1N hydrochloric acid to 900 g of water, 0.25 g of commercially available β-mannanase derived from Aspergillus bacteria and 100 g of guar gum powder were added and mixed. This mixture was allowed to react at 50° C. to 55° C. for 12 hours. Thereafter, the enzyme was inactivated by heating at 90° C. for 15 minutes. The reaction solution was filtered and separated by suction filtration, and the transparent solution obtained by removing insoluble matter was concentrated under reduced pressure (Yamato evaporator). A solid content of 20% by mass was obtained. This transparent solution was dried using a spray dryer (Okawahara Kakoki Co., Ltd.), yielding 70 g of partially hydrolyzed guar gum as a powder.

The average molecular weight of the guar gum decomposition product was determined in the same manner as in Example 1 and was approximately 1.5×10⁴. The HPLC chart showed that 80% by mass or more of the components of the PHGG had a molecular weight in the range from 1.0×10³ to 2.0×10⁵.

The viscosity of a 1% by mass aqueous solution was measured using a B-type viscometer at 25° C. and 60 rpm, and was found to be 9 mPa·s.

The galactose to mannose content ratio (galactose:mannose) was measured to be 1:2.0.

The dietary fiber content was measured using enzymatic HPLC, and was found to be 88% by mass.

### Test Method

A placebo-controlled, double-blind, randomized, parallel-group comparative study was conducted to verify the effects of PHGG on elderly people. 91 healthy subjects age 60 years or older were recruited, and 22 subjects who did not meet the selection criteria and 3 subjects who declined to participate were excluded. The remaining 66 subjects were randomly divided into two groups of 33 subjects each. One group consumed placebo (powdered dextrin) every day for 12 weeks; the other group consumed 5 g/day of PHGG from Example 1 every day for 12 weeks. However, since there was one subject in the PHGG group who did not receive the allocation at the start or during the test period and there were subjects who discontinued participation (2 in the placebo group and 3 in the PHGG group), statistical analysis was performed on 31 subjects in the placebo group and 30 subjects in the PHGG group.

Ten types of tests (verbal memory test, visual memory test, finger tapping test, socially distributed cognition (SDC) test, Stroop test, attention shifting test, sustained processing test, facial expression recognition test, logical thinking test, and four-part sustained processing test) were administered using Cognitrax® software at week 0 and week 12. From these tests, standardized scores were obtained for each cognitive domain (Neurocognition Index (NCI), composite memory, verbal memory, visual memory, psychomotor speed, reaction time, complex attention, cognitive flexibility, processing speed, executive function, social acuity, reasoning, working memory, sustained attention, simple attention, and motor speed).

### Results

Table 1 shows a summary of the cognitive function tests from mainly results of visual memory. The PHGG group showed higher scores than the placebo group after 12 weeks (PHGG group: 99.6±15.8, placebo group: 90.5±15.4, difference between groups: 9.1, P=0.023).

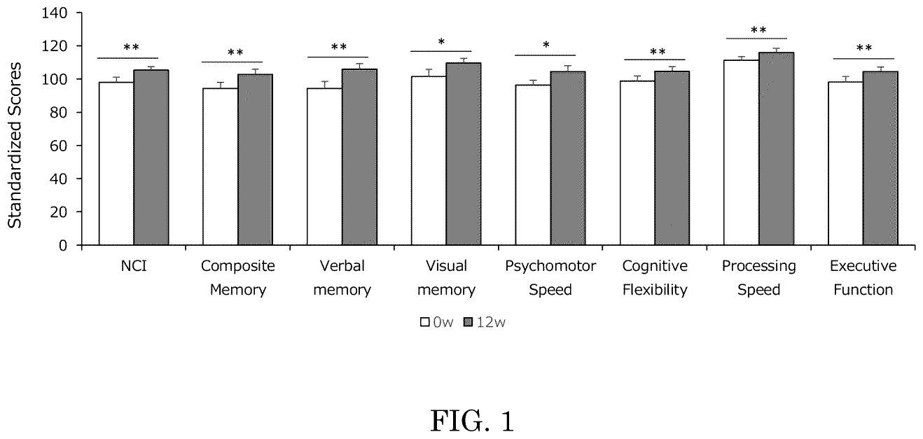

FIG. 1 shows a comparison of data concerning eight cognitive domains (NCI, composite memory, verbal memory, psychomotor speed, reaction time, cognitive flexibility, processing speed, and executive function) that showed significant differences between the PHGG group before the start of the test (week 0) and after the test (week 12) among the 16 standardized scores mentioned above. Notable improvements were observed following PHGG administration.

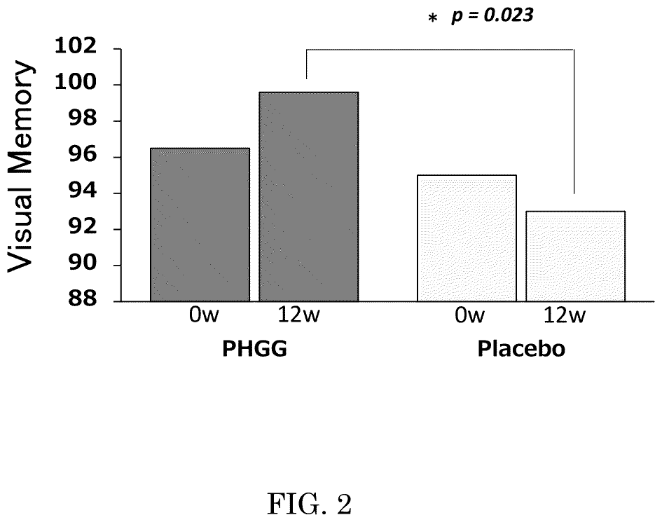

FIG. 2 shows the results of visual memory, where a significant difference was found between the placebo group and the test food group.

TABLE 1

| | | Baseline | Week 12 |
|---|---|---|---|
| Neurocognition index | Placebo (n = 30) | 89.5 ± 16.5 | 100.9 ± 11.1 |
| (NCI) | PHGG (n = 31) | 98.1 ± 16.4 | 105.4 ± 11.2 |
| | p-value | 0.048 | 0.115 |
| Composite memory | Placebo (n = 30) | 87.6 ± 21.4 | 96.5 ± 17.4 |
| | PHGG (n = 31) | 94.3 ± 19.9 | 102.9 ± 16.9 |
| | p-value | 0.211 | 0.147 |
| Verbal memory | Placebo (n = 30) | 86.5 ± 23.9 | 103.5 ± 19.0 |
| | PHGG (n = 31) | 94.4 ± 23.2 | 105.8 ± 19.0 |
| | p-value | 0.198 | 0.646 |
| Visual memory | Placebo (n = 30) | 93.1 ± 17.9 | 90.5 ± 15.4 |
| | PHGG (n = 31) | 96.5 ± 13.7 | 99.6 ± 15.8 |
| | p-value | 0.418 | 0.026 |
| Psychomotor speed | Placebo (n = 30) | 87.1 ± 32.9 | 105.1 ± 17.1 |
| | PHGG (n = 31) | 101.4 ± 23.9 | 109.6 ± 15.1 |
| | p-value | 0.058 | 0.278 |
| Reaction time | Placebo (n = 30) | 101.8 ± 28.9 | 100.7 ± 21.4 |
| | PHGG (n = 31) | 96.3 ± 16.5 | 104.5 ± 18.7 |
| | p-value | 0.367 | 0.464 |
| Complex attention | Placebo (n = 30) | 81.5 ± 40.4 | 100.1 ± 23.4 |
| | PHGG (n = 31) | 99.5 ± 26.5 | 105.2 ± 24.4 |
| | p-value | 0.044 | 0.405 |
| Cognitive flexibility | Placebo (n = 30) | 89.9 ± 16.8 | 101.9 ± 13.8 |
| | PHGG (n = 31) | 98.6 ± 18,2 | 104.8 ± 14.8 |
| | p-value | 0.057 | 0.442 |
| Processing speed | Placebo (n = 30) | 106.7 ± 14.2 | 112.0 ± 11.9 |
| | PHGG (n = 31) | 111.3 ± 12.3 | 115.9 ± 14.0 |
| | p-value | 0.186 | 0.246 |
| Executive function | Placebo (n = 30) | 91.7 ± 15.7 | 102.8 ± 12.3 |
| | PHGG (n = 31) | 98.2 ± 18.3 | 104,4 ± 15.2 |
| | p-value | 0.146 | 0.654 |
| Social acuity | Placebo (n = 30) | 79.3 ± 26.4 | 89.7 ± 22.0 |
| | PHGG (n = 31) | 91.0 ± 23.7 | 91.6 ± 20.3 |
| | p-value | 0.073 | 0.733 |
| Reasoning | Placebo (n = 30) | 91.6 ± 14.4 | 97.7 ± 15.7 |
| | PHGG (n = 31) | 99.7 ± 15.4 | 97.4 ± 18.9 |
| | p-value | 0.038 | 0.956 |
| Working memory | Placebo (n = 30) | 103.8 ± 13.4 | 106.3 ± 15.6 |
| | PHGG (n = 31) | 108.9 ± 11.5 | 107.9 ± 12.7 |
| | p-value | 0.118 | 0.654 |
| Sustained attention | Placebo (n = 30) | 100.2 ± 20.7 | 105.8 ± 20.9 |
| | PHGG (n = 31) | 109.9 ± 17.0 | 110.5 ± 10.3 |
| | p-value | 0.052 | 0.272 |
| Simple attention | Placebo (n = 30) | 38.0 ± 170.2 | 76.9 ± 88.7 |
| | PHGG (n = 31) | 84.8 ± 100.9 | 88.2 ± 68.6 |
| | p-value | 0.198 | 0.581 |
| Motor speed | Placebo (n = 30) | 78.6 ± 39.9 | 99.3 ± 17.6 |
| | PHGG (n = 31) | 94.5 ± 27.9 | 102.3 ± 15.0 |
| | p-value | 0.077 | 0.476 |

These results show that cognitive functioning (particularly visual memory after 12 weeks) was significantly improved in the PHGG group compared to the placebo group.

In addition, it was found that composite memory, verbal memory, psychomotor speed, reaction time, cognitive flexibility, and executive function were significantly improved in the PHGG group after 12 weeks of intake.

Additional aspects of the present teachings include, but are not limited to:

1. A cognitive function decline inhibitor (promotor) for a healthy subject comprising a partially hydrolyzed guar gum (PHGG) having an average molecular weight of 1.0×10³ to 2.0×10⁵, wherein 70% or more by mass of the components of the PHGG fall within that range, and the viscosity of a 1% by mass aqueous solution of the PHGG measured at 25° C. and 60 rpm using a B-type viscometer, of 50 mPa·s or less. The partially hydrolyzed guar gum is preferably produced by hydrolyzing galactomannan polysaccharide, which is obtained from guar endosperm and has a galactose to mannose content ratio (galactose:mannose) in the range of 1:1.3 to 1:2.1, using a microbial β-mannanase to reduce the molecular weight. The partially hydrolyzed guar gum preferably contains at least 70% by mass of dietary fiber as determined by enzymatic HPLC.

2. The cognitive function decline inhibitor as defined in the above Aspect 1, wherein the healthy subject is 60 years old or older.

3. The cognitive function decline inhibitor as defined in the above Aspect 1 or 2, wherein the (at least one) cognitive function is selected from visual memory, composite memory, verbal memory, psychomotor speed, reaction time, cognitive flexibility, and executive function.

4. A food or drink comprising the cognitive function decline inhibitor as defined in any one of the above Aspects 1-3.

5. A pharmaceutical composition comprising the cognitive function decline inhibitor as defined in any one of the above Aspects 1-3.

6. The pharmaceutical composition as defined in the above Aspect 5, further comprising a pharmaceutically acceptable excipient and/or filler.

7. The pharmaceutical composition as defined in the above Aspect 6, wherein the pharmaceutically acceptable excipient and/or filler comprises one or more compounds selected from the group consisting of calcium carbonate, dicalcium phosphate, dry starch, calcium sulfate, cellulose, compressible sugars, confectioner's sugar, dextrate, dextrin, dextrose, dibasic calcium phosphate dihydrate, glyceryl palmitostearate, hydrogenated vegetable oil (type I), inositol, kaolin, lactose, magnesium carbonate, magnesium oxide, maltodextrin, mannitol, microcrystalline cellulose, polymethacrylates, potassium chloride, powdered cellulose, powdered sugar, pregelatinized starch, sodium chloride, sorbitol, starch, sucrose, sugar spheres, talc, tribasic calcium phosphate, and mixtures thereof. Preferably, the pharmaceutically acceptable excipient and/or filler is selected from the group consisting of dicalcium phosphate, cellulose, compressible sugars, dibasic calcium phosphate dihydrate, lactose, mannitol, microcrystalline cellulose, starch, tribasic calcium phosphate, and mixtures thereof.

8. The pharmaceutical composition as defined in any one of the above Aspects 5-7, wherein the pharmaceutical composition is contained in a tablet, pill, suppository, granules or powder, syrup or other types of liquids, etc. Tablets may be prepared according to methods known in the art, including dry granulation (e.g., roller compaction), wet granulation (e.g., fluid bed granulation and high shear granulation), and direct compression, and the type of excipients and/or fillers used will vary accordingly. The tablets, pills, etc. may be packaged, e.g., in a blister pack or bottle.

1. A method for promoting at least one cognitive function in a human in need thereof, comprising:
enterally administering to the human a therapeutically effective amount of a partially hydrolyzed guar gum having an average molecular weight in a range from 1.0×10³ to 2.0×10⁵,
wherein:
70% or more by mass of the components of the partially hydrolyzed guar gum fall within the range from 1.0×10³ to 2.0×10⁵, and
a 1% by mass aqueous solution of the partially hydrolyzed guar gum has a viscosity, measured at 25° C. and 60 rpm using a B-type viscometer, of 50 mPa·s or less.

2. The method of claim 1, wherein the partially hydrolyzed guar gum:
is produced by hydrolyzing galactomannan polysaccharide, which is obtained from guar endosperm and has a galactose to mannose content ratio (galactose:mannose) in the range of 1:1.3 to 1:2.1, using a β-mannanase, and
contains at least 70% by mass of dietary fiber as determined by enzymatic HPLC.

3. The method of claim 1, wherein the human is age 60 years or older.

4. The method of claim 1, wherein the at least one cognitive function is selected from the group consisting of visual memory, composite memory, verbal memory, psychomotor speed, reaction time, cognitive flexibility, and executive function.

5. The method of claim 1, wherein the at least one cognitive function is visual memory.

6. The method of claim 1, wherein the therapeutically effective amount of the partially hydrolyzed guar gum is in the range of from 0.5 g to 70 g per day.

7. The method of claim 1, wherein the therapeutically effective amount of the partially hydrolyzed guar gum is in the range of from 3 g to 30 g per day.

8. The method of claim 1, wherein the therapeutically effective amount of the partially hydrolyzed guar gum is in the range of from 3 g to 18 g per day.

9. The method of claim 1, wherein 80% by mass or more of the components of the partially hydrolyzed guar gum have a molecular weight in a range from 1.0×10³ to 2.0×10⁵.

10. The method of claim 1, wherein the partially hydrolyzed guar gum has a molecular weight in a range from 2.0×10³ to 1.0×10⁵.

11. The method of claim 1, wherein the partially hydrolyzed guar gum has a molecular weight in a range from 2.0×10³ to 2.5×10⁴.

12. The method of claim 1, wherein the partially hydrolyzed guar gum is administered orally.

13. The method of claim 12, wherein the partially hydrolyzed guar gum has a molecular weight in a range from 2.0×10³ to 1.0×10⁵.

14. The method of claim 12, wherein the partially hydrolyzed guar gum has a molecular weight in a range from 2.0×10³ to 2.5×10⁴.

15. The method of claim 14, wherein 80% by mass or more of the components of the partially hydrolyzed guar gum have a molecular weight in a range from 1.0×10³ to 2.0×10⁵.

16. The method of claim 15, wherein the therapeutically effective amount of the partially hydrolyzed guar gum is in the range of from 3 g to 30 g per day.

17. The method of claim 16, wherein the at least one cognitive function is selected from the group consisting of visual memory, composite memory, verbal memory, psychomotor speed, reaction time, cognitive flexibility and executive function.

18. The method of claim 16, wherein the at least one cognitive function is visual memory.
